(19) United States
(12) Patent Application Publication
(10) Pub. No.: US 2008/0166335 A1
(43) Pub. Date: Jul. 10, 2008

(54) Slimming Cosmetic Composition Comprising a Metalloproteinase as an Active Agent

(76) Inventor: Olivier Courtin, Boulogne-Billancourt (FR)

Correspondence Address: DAVID R PRESTON & ASSOCIATES APC, 5850 OBERLIN DRIVE, SUITE 300, SAN DIEGO, CA, 92121, US

(21) Appl. No.: 10/592,457

(22) PCT Filed: Mar. 8, 2005
(86) PCT No.: PCT/FR2005/000554

§ 371 Date: Oct. 26, 2006

(30) Foreign Application Priority Data
Mar. 8, 2004 (FR) 0450461

(51) Int. Cl. A61K 38/48; A61Q 19/00

(52) U.S. Cl. 424/94.67

(57) ABSTRACT

The invention relates to a slimming cosmetic composition comprising, by way of an active agent, at least one inhibitor of metalloproteinases −2 and/or −9 or a plant extract containing said metalloproteinase inhibitor. The invention also relates to the use of an inhibitor of metalloproteinases −2 and/or −9 or a plant extract containing said inhibitor in a slimming cosmetic product. The invention further relates to the cosmetic use of an inhibitor of metalloproteinases −2 and/or −9 or a plant extract containing said inhibitor as an agent that prevents adipocyte differentiation for the preparation of a slimming composition.

The present invention concerns the field of cosmetics and more specifically the field of slimming preparations. The present invention also concerns a cosmetic slimming preparation comprising a matrix metalloproteinase (MMP) inhibitor 2 and/or 9. The present invention also concerns compositions which are useful in regulating adipocyte differentiation, thereby reducing lipogenesis.

Adipose tissue (or the hypodermis) is attached to the lower part of the dermis by expansions of collagen fibre. Its thickness is variable; thin on the forehead, it develops broadly on particular areas (thighs, abdomen). The anatomical localisation of adipose tissue is a genuine secondary sexual characteristic. In men, it predominates above the waist, on the abdomen and shoulders, whereas in women, it is concentrated below the waist, in the lower part of the abdomen and on the hips, buttocks and thighs. This sex-related localisation is heavily pronounced in case of obesity, with a distinction being drawn between two forms, android and gynoid obesity.

Adipose tissue is comprised of:
specific cells, adipocytes, which are able to store fats (lipogenesis) but also mobilise the latter (lipolysis),
an extracellular matrix,
a dense network of capillaries.

Development of adipose tissue is characterised by modification of all the components of the hypodermal tissue. These disturbances also have repercussions on the more superficial layer. In the dermis, an increase in the thickness, probably due to fluid engorgement (water retention) and infiltration of adipose lobules distorting the junction between the dermis/hypodermis are observed; the surface of the skin acquires a padded appearance, which is the notorious “orange peel effect”.

Development of adipose tissue involves various different mechanisms:
reorganisation of the extracellular matrix,
differentiation of the pre-adipocytes into adipocytes
increase in the volume of the adipocytes.

The extracellular matrix structures organisation of the hypodermis; it gives it a certain firmness. It is mainly made up of collagen, elastin, proteoglycans, fibronectin and other glycosylated proteins. These macromolecules are organised in a complex fibrillated network. The degradation mechanisms which transform this network occur owing the action of one or several members of the metalloproteinase family. Some twenty metalloproteinases have been identified to date. In the hypodermis, the metalloproteinases 2 and 9 are found (two gelatinases) secreted by the adipocytes (A. Bouloumié, C. Sengenés, G. Portolan, J. Galitzky and M. Lafonat, Diabetes, 2001, vol. 50, pages 2080-2086). The metalloproteinases 2 and 9 degrade the surrounding extracellular matrix, thereby contributing to the dissolution of its architecture. The extracellular matrix disorganise in this manner makes way for the adipocytes which reorganise the tissue, occupy the space and promote emergence of a new capillary network necessary for their development.

It is now well established that the number of adipocytes may evolve during life. In the adipose tissue, the presence of “precursor” cells known as preadipocytes has been demonstrated. These cells are able, under the influence of stimuli (insulin and glucose, chemokines secreted by mature adipocytes) of transforming themselves into adipocytes (cells capable of storing fats): this is adipocyte differentiation. Increase in the number of adipocytes (or tissue hyperplasia) is therefore an important element in development of fatty tissue. Reorganisation of the extracellular matrix and adipocyte differentiation are very closely linked: the addition of metalloproteinase-inhibiting molecules to preadipocyte cells allows inhibition of their differentiation and therefore limitation of development of fatty tissue. This involves a new therapeutic approach in management of cellulitis which acts by limiting development of the fatty tissue and by protecting the conjunctive tissue of the hypodermis.

Adipocytes are also able to increase their own storage ability by increasing the quantity of fats stored. Fatty acid uptake by adipocytes is performed by means of an enzyme secreted by the adipocytes: the lipoprotein lipase. The lipoprotein lipase hydrolyses the triglycerides transported by blood lipoproteins. In the adipocyte, the fatty acids are esterified by means of a glucose metabolite and are essentially stored in the form of triglycerides. This stage requires the presence of an enzyme, glycerol 3 phosphate dehydrogenase (G3PDH).

In contrast, the adipocytes are also capable of mobilising the stored fats; this is lipolysis. Lipolysis is under the control of a hormone, lipase sensitive to hormones and splits the triglycerides into fatty acids and glycerol and allows their elimination from the cell. Lipase sensitive to hormones being dependent on the intracellular AMP_{c} and GMP_{c}, consequently an increase of AMP_{c} and GMP_{c} levels promote lipolysis.

Many cosmetic slimming preparations were proposed in the former art which promoted lipolysis and/or inhibited lipogenesis. One may mention, more specifically, the cosmetic slimming preparations described in the French patent of the applicant published under no. 2 729 856. This patent proposes cosmetic slimming preparations capable, by means of soluble organic substances, or enzymes, of refining and preparing the skin to receive active substances the effects of which are increased in order to promote elimination of the lipids and prevent storage of these lipids.

The applicant's research allowed identification of the presence of metalloproteinase 2 and/or 9 inhibitors in plant extracts and therefore envisage slimming compositions containing the aforementioned inhibitors or the aforementioned extracts.

The present invention therefore relates to a cosmetic slimming preparation characterised by the fact that it comprises, as the active substance, at least one metalloproteinase 2 and/or 9 inhibitor, preferably in the form of a plant extract containing this inhibitor as a protector of the extracellular matrix and intended to prevent adipocyte differentiation.

The composition according to the invention may contain one or several plant extracts including a. metalloproteinase 2 and/or 9 inhibitor. More specifically, the composition according to the invention may contain an extract of Baccharis genistelloides. This herb from Amazonia belongs to the asteracea family.

Advantageously, the extract useable within the context of the present invention is derived from the aerial parts of Baccharis genistelloides and preferably, a hydroglycolic extract is involved. After dissolution of Baccharis genistelloides in a water/butylene glycol mixture (50/50), the soluble and insoluble phases are separated by filtration. Subsequently, sterilising filtration of the soluble phase is performed in order to obtain the final extract of Baccharis genistelloides which appears in the form of an amber coloured clear liquid with a characteristic odour. It has the following analytical characteristics:
pH: 5 to 6
dry matter: 13 to 22 g/l
total polyphenols (expressed as gallic acid): 0.7 to 1.4.

Apart from the plant extract containing at least one metalloproteinase 2 and/or 9 inhibitor, the composition of the invention may include at least one active substance useful in order to inhibit lipogenesis or promote lipolysis or furthermore in order to refine, tone and lift the surface of the skin or hydrate the latter. This active substance is preferably caffeine, which has a lipolytic action. Furthermore, the composition of the invention may include at least one active substance selected from among the following compounds and extracts:
an extract of sweet broom or butcher's broom or Ruscus, which is rich in mineral elements such as calcium and potassium, in addition to saponosides and sapogenins. The presence of saponosides gives this extract the following properties:
anti-inflammatory, calming and soothing,
lightening, effective for protection against redness,
promoting microcirculation and therefore improving capillary tonicity,
skin decongestant.
one or several silicon derivatives, such as SILANOL, which has the following properties:
moisturising, allowing regularisation of the fluid concentration,
prevention and regeneration, particularly anti-radical activity, which allows reorganisation of membrane lipids by making the membrane more resistant to attack by free radicals,
anti-inflammatory, which reduces vasodilation and cancels out the action of arachidonic acid.
an extract of Indian chestnut, which has the following properties:
vitamin P, which increases resistance and reduces permeability of the blood capillaries (vasoprotector),
decongestant and soothing properties against inflammation, since the saponin derivatives of Indian chestnut make it possible to treat the “orange peel” effect.
an extract of Ginkgo Biloba, the dry leaves of which are used in medicine as a venotonic owing to their flavonoid content.
an extract of red vine. Red vine leaves are rich in flavonoids and allow reinforcement of the vessel walls and venous tone. This type of extract therefore acts on microcirculation.

The proportion of the plant extract containing at least one metalloproteinase 2 and/or 9 inhibitor in the composition according to the invention is between 0.1 to 10%, preferably on the order of 0.1 to 5% (weight/weight).

The composition according to the invention may furthermore comprise one or several formulation agents or additives, the use of which is known and conventional in cosmetic and dermatological compositions such as, as an example and in a non-limitative manner, soothing agents, colorants, filmogenic active substances, surfactants, preservatives, emulsifiers, oils, glycols and vitamins, etc. Owing to this knowledge in the field of cosmetics, a professional will know which formulation agents to add to the composition of the invention and in which quantities as a function of the desired properties.

The composition of the invention may come in any form known to professionals in the cosmetics sector, such as for example, a cream, milk, lotion, gel, mask, etc. . . . , without any particular restriction in terms of pharmaceutical form apart from those for application to the skin.

The present invention also concerns use of a metalloproteinase 2 and/or 9 inhibitor as defined above in a cosmetic slimming product. Furthermore, the present invention concerns cosmetic use of a metalloproteinase 2 and/or 9 inhibitor as defined above as an agent to prevent adipocyte differentiation, for preparation of a slimming composition.

The examples below are given as an illustration and must not be interpreted as restricting the scope of the invention.

They concern on the one hand, demonstration of the role of an extract of Baccharis genistelloides on inhibition of adipocyte differentiation and on inhibition of metalloproteinase 2 and 9 secretion and their activity and furthermore, examples of the compositions which are the subjects of the present invention.

The examples make reference to the following figures in which:

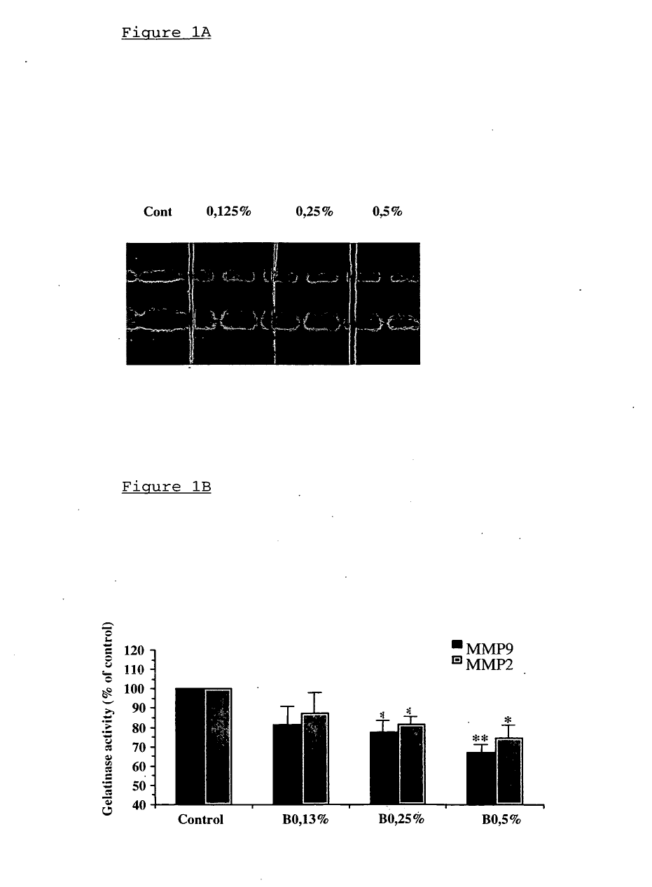

FIG. 1 represents the effects of the extract of Baccharis genistelloides on activity of the adipocyte metalloproteinases MMP-2 and MMP-9 (n=3). The effects of the product were tested on the activity of the metalloproteinases MMP-2 and MMP-9 present in culture supernatants of preadipocytes differentiated 5 days. (A) Photography of representative gels. (B) Quantification of the zymographies. * p·0.05 or ** P·0.01 treated vs. control.

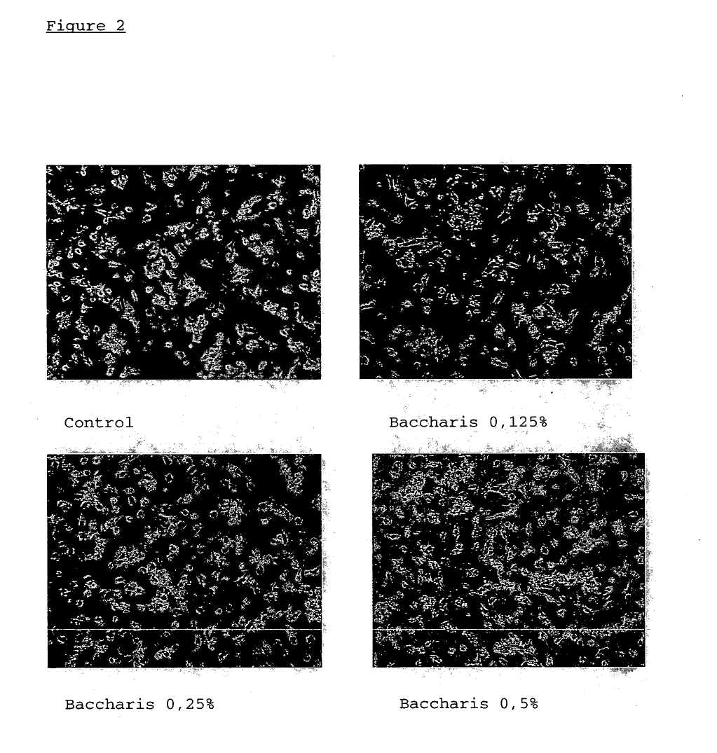

FIG. 2 represents the preadipocytes differentiated 10 days in the presence of the extract of Baccharis genistelloides (0.125%, 0.25% or 0.5%).

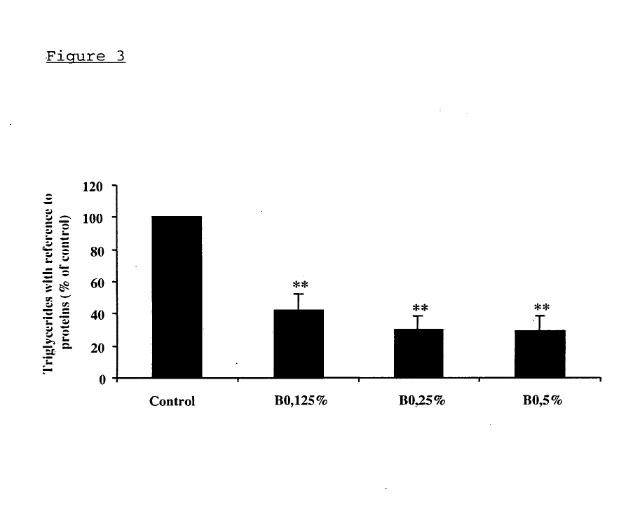

FIG. 3 represents the effects of an extract of Baccharis genistelloides on the intracellular triglyceride levels in the preadipocytes (n=4). The preadipocytes differentiated 10 days in the presence of the extract of Baccharis genistelloides (0.125%, 0.25% or 0.5%). The triglyceride levels were related to the protein levels and are expressed in percentage of the control. * P·0.05, ** P·0.01 treated vs. control.

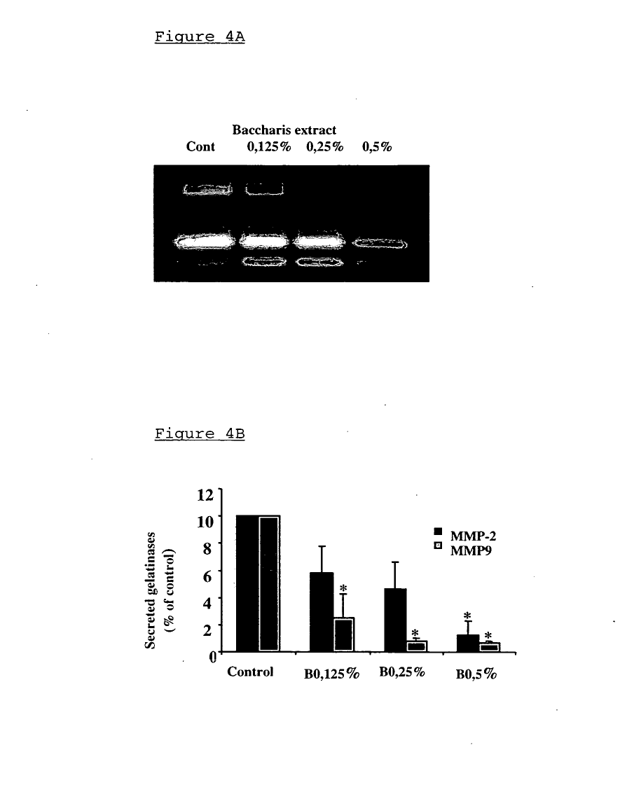

FIG. 4 represents the effects of an extract of Baccharis genistelloides on secretion of the gelatinases MMP-2 and MMP-9 by the preadipocytes (n=4). The gelatinase activities were measured in the culture supernatants by zymography with gelatine and are expressed in % of the control. (A) Photograph ** P·0.01 treated vs. control. (B) Quantification of the zymographies.

### I. Activity of the Extract of Baccharis Genistelloides.

### A. Equipment and Methods.

1. Culture and processing of the human preadipocytes.

The subcutaneous adipose tissue is derived from overweight or moderately obese individuals having undergone plastic surgery (abdominal dermolipectomy). After digestion with collagenase under agitation, the cells of the stroma-vascular fraction are inoculated into Eagle medium modified by Dulbecco (DMEM)/F12 supplemented with 10% of foetal calf serum (SCS) and a mixture of antibiotics (biotin 33 μM, D-pantothenate 17 μM, gentamycine 50 μg/ml) and filtered again (70 μm filter). The cells are incubated at 37° C. in an atmosphere of 95% of air, 5% of CO₂. After 24 h, the medium is replaced by DMEM/F12 to which antibiotics and hormones have been added: insulin 22% M, transferrin 10 μg/ml, cortisol 100 μM, T3 2 μM in addition to ciglitizone 1 mg/ml (a PPAR agonist) for 72 hours. After these 72 hours, the medium is replaced with the same medium without ciglitizone, with or without the extract of Baccharis genistelloides (B 0.125%, B 0.25%, B 0.5%).

This day is considered as D0. This differentiating medium is renewed every two days. After 10 days of differentiation, the supernatants are collected and stored at −20° C. until the assays and analyses by zymography. The cells are frozen at −20° C. after washing with PBS.

2. Assays of proteins and triglycerides.

The cells are collected in 100 μL of PBS, lysed by sonication and the quantities of intracellular proteins and triglycerides are assayed using the DC Bio-Rad Protein Assay and GPO-Trinder kits from Bio-Mérieux.

3. Zymography with gelatine.

The proteins with gelatinase activity (MMP-2 and MMP-9) are identified by electrophoresis on SDS-polyacrylamide gels (SDS-PAGE) containing a substrate of the MMP's: the gelatine (1 mg/ml) (ICN Biomedicals).

a—in order to determine the effect of the Baccharis genistelloides extract on secretion of MMP's by the cells, the culture medium of the cells treated with the agents (20 μl +10 μl of non-denaturing deposit blue is applied directly to the gels. Following electrophoresis, the proteins are renatured by incubating the gels with 2.5% of Triton x-100 (incubation: twice 15 min). The gels are subsequently incubated for 16 hours at 37° C. in 50 mmol/L of Tris-HCl (pH 8.5), 0.02% of NaN₃ and 5 mmol/L of CaCl2. At the end of incubation, the gels are coloured with Coomassie blue and the gelatinase activity is visualised by regions of lysis on the coloured gel.

b—In order to determine the effect of the extract of Baccharis genistelloides on the activity of the MMP's, the culture medium of the control cells differentiated for 5 day (20 μl +10 μl of non-denaturing deposit blue (1 mol/1 Tris HCl, pH 6.8, glycerine, Sodium Dodecyl Sulphate 20%, β mercaptoethanol, Bromophenol Blue 0.05%) is applied directly into the gels. Following electrophoresis, the proteins are renatured by incubating the gels with 2.5% of Triton X-100 (incubation: twice 15 min). The gels are subsequently incubated for 16 hours at 37° C. in 50 mmol/L of Tris-HCl (pH 8.5), 0.02% of NaN₃ and 5 mmol/L of CaCl2 in the absence or in the presence of the extract of Baccharis genistelloides (B 0.125%, B 0.25%, B 0.5%). At the end of incubation, the gels are coloured with Coomassie Blue (50% methanol, 10% of acetic acid, 0.1% of Coomassie Brilliant Blue, 40% of distilled water) and the gelatinase activity is visualised by regions of lysis on the coloured gel.

4. Statistical analyses.

The values yielded are expressed as a percentage of the control±standard deviation from the mean (sdm) for (n) independent experiments. The statistical analyses are performed by one-factor variance analyses (ANOVA) followed by a post hoc Dunnett test. The values are considered significant when p<0.05.

### B. Results.

1. Effect of the extract of Baccharis genistelloides on the activity of the MMP's.

We studied the effect of the extract of Baccharis genistelloides on the activity of the MMP's secreted by human preadipocytes differentiated for 5 days (MMP-2 and MMP-9). FIG. 1 shows that the extract of Baccharis genistelloides inhibits the activity of MMP-2 and MMP-9 in a dependent manner from the concentration.

2. Effect of the EXTRACT of Baccharis genistelloides on differentiation of human preadipocytes in primary culture.

We processed human preadipocytes derived from 2 different abdominal dermolipectomies (ADL) with the extract of Baccharis genistelloides.

FIG. 2 shows the effects of the product on the morphology of the human preadipocytes in primary culture after 10 days of treatment. One observes a reduction in the quantity of fluid droplets in the treated cells in relation to the control cells.

We were able to observe a significant reduction in the intracellular triglyceride content (with reference to mg of proteins) in all cases (FIG. 3).

We finally studied the effect of the Baccharis genistelloides extract on secretion of MMP-2 and MMP-9 by the preadipocytes and we showed (FIG. 4) a significant reduction in secretion of both proteases with treatment.

### II. Examples of Compositions Which are the Subject of the Invention

| Demineralised water | q.s.p 100 |
|---|---|
| Pemulen tri | 0.5 |
| Glycerine | 5.0 |
| Anhydrous caffeine | 2.0 |
| Pob butyl | 0.05 |
| Silicone oil | 7.5 |
| C12-C15 alkyl benzoate | 1.5 |
| Extract of Baccharis genistelloides | 1.0 |
| Silicon derivative | 3.0 |
| Extract of geranium robertianum | 0.5 |
| Tea | 0.5 |
| Denatured alcohol at 96° | 15.0 |
| Menthol | 0.35 |
| Perfume | 0.6 |
| Colorants at 1% | 0.12 |

| | |
|---|---|
| Vegetable oil | 1.0 |
| Silicone oil | 15.0 |
| Isononyl isononanoate | 10.0 |
| Cetyl alcohol | 1.0 |
| Cetearyl glucoside | 5.0 |
| Glycerine | 3.0 |
| Sepigel 305 | 0.5 |
| Anhydrous caffeine | 2.0 |
| Extract of Baccharis genistelloides | 1.0 |
| Extract of gingko biloba | 1.0 |
| Extract of red vine | 1.0 |
| Menthol | 0.25 |
| Phenonip | 0.8 |
| Potassium sorbate | 0.1 |
| Perfumes | 0.5 |

| Demineralised water | q.s.p 100 |
|---|---|
| Permulen TR1 | 0.2 |
| Carbomer | 0.4 |
| Anhydrous caffeine | 2.0 |
| Extract of butcher's broom | 1.0 |
| Extract of Indian chestnut | 1.0 |
| Extract of Baccharis genistelloides | 1.0 |
| Tea | 0.65 |
| Denatured alcohol at 96° | 15.0 |
| Menthol | 0.4 |
| Perfume | 0.4 |

1) Use of a plant extract comprising at least one metalloproteinase 2 and/or 9 inhibitor as the active substance for preparation of a cosmetic slimming composition.

2) Use of a plant extract according to claim 1, characterised by the fact that the aforementioned plant extract is an extract of Baccharis genistelloides.

3) Use of a plant extract according to claim 1 or 2, characterised by the fact that the aforementioned extract is a hydro-glycolic extract of the plant.

4) Use of a plant extract according to any of the preceding claims, characterised by the fact that the aforementioned cosmetic slimming composition further comprises at least one active agent useful in order to inhibit lipogenesis or promote lipolysis or furthermore to refine, tone and lift the surface of the skin or moisturise the latter.

5) Use of a plant extract according to claim 4, characterised by the fact that the aforementioned cosmetic slimming composition includes caffeine.

6) Use of a plant extract according to any of claims 4 and 5, characterised by the fact that the aforementioned cosmetic slimming composition comprises at least one active agent selected from among butcher's broom, a silicon derivative, an extract of Indian chestnut, an extract of gingko biloba or an extract of red vine.

7) Use of a plant extract according to any of the preceding claims, characterised by the fact that the proportion of plant extract containing at least one metalloproteinase 2 and/or 9 inhibitor in the aforementioned cosmetic slimming composition is between 0.1 and 10% and preferably 0.1 to 5%.

8) Cosmetic slimming care process consisting in applying to the skin a plant extract comprising at least one metalloproteinase 2 and/or 9 inhibitor or a composition containing this plant extract.

9) Cosmetic slimming care process according to claim 8, characterised by the fact that the aforementioned plant extract is an extract of Baccharis genistelloides.

10) Cosmetic slimming care process according to claim 8 or 9, characterised by the fact that the aforementioned plant extract is a hydro-glycolic extract of the plant.

11) Cosmetic slimming care process according to claims 8 to 10, characterised by the fact that the aforementioned composition comprises in addition an active agent useful in inhibiting lipogenesis or in promoting lipolysis or furthermore in refining, toning and lifting the surface of the skin or in moisturising the latter.

12) Cosmetic slimming care process according to claim 11, characterised by the fact that the aforementioned composition contains caffeine.

13) Cosmetic slimming care process according to any of claims 11 and 12, characterised by the fact that the aforementioned composition comprises at least one active agent selected from among extract of butcher's broom, a silicon derivative, an extract of Indian chestnut, an extract of ginkgo biloba or an extract of red vine.

14) Cosmetic slimming care process according to any of claims 8 to 13, characterised by the fact that the proportion of plant extract containing at least one metalloproteinase 2 and/or 9 inhibitor in the aforementioned composition is between 0.1 and 10% and preferably 0.1 to 5%.
